(19) United States
(12) Patent Application Publication
(10) Pub. No.: US 2012/0003666 A1
(43) Pub. Date: Jan. 5, 2012

(54) METHOD OF DETECTING TARGET SUBSTANCE

(75) Inventor: Masaaki Kobayashi, Kawasaki-shi (JP)

(73) Assignee: CANON KABUSHIKI KAISHA, Tokyo (JP)

(21) Appl. No.: 12/744,047

(22) PCT Filed: Dec. 10, 2008
(86) PCT No.: PCT/JP2008/072900

§ 371 Date: May 20, 2010

(30) Foreign Application Priority Data
Dec. 20, 2007 (JP) 2007-328716

(51) Int. Cl. G01N 33/82; G01N 21/55; G01N 21/59; G01N 33/53

(52) U.S. Cl. 435/7.5; 436/501

(57) ABSTRACT

Provided is a method of measuring a being state of a target substance at unknown concentration contained in a sample. The method includes identifying a kind of a target substance when one kind of target substance is contained in a sample and determining a concentration ratio when plural kinds of target substances are contained in the sample: by obtaining, with respect to each of plural kinds of known substances having the same recognition site, a relationship between Parameter A of the known substance and Parameter B of the known substance; and measuring Parameter A of the target substance contained in the sample and Parameter B of the target substance contained in the sample, wherein Parameter A of X is a value derived from a number of molecules of X and Parameter B of X is a value derived from the number and a molecular weight of X.

### TECHNICAL FIELD

The present invention relates to a method of detecting a target substance.

### BACKGROUND ART

In recent years, measuring the number of molecules and the molecular weight of each of plural target substances recognizing the same substance has been demanded.

Adiponectin which is a hormone secreted from adipocytes has bioactivities such as an antiarteriosclerotic effect and an insulin resistance-ameliorating effect, and hence the adiponectin is attracting attention as one of the risk factors of diabetes and coronary artery diseases. It is suggested that the adiponectin is present in blood in several being states (low-molecular-weight, middle-molecular-weight, and high-molecular-weight), and pathosis of metabolic syndrome can be diagnosed more accurately by abundance of the high-molecular-weight adiponectin. Therefore, it is important to measure the being state of the adiponectin, that is, a molecular weight of the adiponectin as well as a concentration of the adiponectin in blood.

With respect to the issue, Japanese Patent Application Laid-Open No. 2006-226959 discloses, for the purpose of measurement and mass analysis of a target substance in a sample, a technology involving an apparatus in which a surface plasmon resonance measuring device and a mass spectrometer are connected. According to the invention, the presence and absence or a concentration of the target substance can be measured by a surface plasmon resonance and a molecular weight of the target substance can be measured by a mass analysis.

However, the invention requires the mass spectrometer, which is an extreme technical apparatus, and hence lacks in convenience of a test.

### DISCLOSURE OF THE INVENTION

According to a method of detecting a target substance of the present invention, a being state of a target substance can be easily detected by detecting the number of molecules and a molecular weight of the target substance present in a sample.

The present invention provides a method of detecting a target substance comprising:

i) obtaining, with respect to each of plural kinds of known substances each having the same recognition site, a relationship a between a concentration of a known substance and Parameter A of the known substance and a relationship b between the concentration of the known substance and Parameter B of the known substance, and a relationship c between Parameter A of the known substance and Parameter B of the known substance from the relationship a and the relationship b, wherein Parameter A of X is a value derived from a number of molecules of X and Parameter B of X is a value derived from the number and a molecular weight of X;

ii) measuring Parameter A of a target substance contained in a sample and Parameter B of the target substance contained in the sample;

iii) from the relationship c in each of the plural kinds of known substances, Parameter A of the target substance, and Parameter B of the target substance,

determining, when one kind of target substance is contained in the sample, to which one of the plural kinds of the known substances the target substance corresponds; and

determining concentration ratios of plural kinds of the known substances which form the target substance contained in the sample when plural kinds of the target substance are contained in the sample.

According to the method of detecting a target substance of the present invention, the number of molecules and the molecular weight of a target substance present in a sample can be detected, and a being state of the target substance in the sample can be easily detected.

Further features of the present invention will become apparent from the following description of exemplary embodiments with reference to the attached drawings.

### BRIEF DESCRIPTION OF THE DRAWINGS

FIGS. 1A, 1B, and 1C are conceptual diagrams illustrating a detection device and a detection method in a first embodiment.

FIG. 2 is a conceptual graph illustrating a relationship between a concentration of a known substance and Parameter A of the known substance in the first embodiment.

FIG. 3 is a conceptual graph illustrating a relationship between the concentration of the known substance and Parameter B of the known substance in the first embodiment.

FIG. 4 is a conceptual graph illustrating a relationship between Parameter A of the known substance and a signal derived from the molecular weight of the known substance in the first embodiment.

FIGS. 5A, 5B, and 5C are conceptual diagrams illustrating a detection device and a detection method in a second embodiment.

FIG. 6 is a conceptual graph illustrating a relationship between a concentration of a known substance and Parameter A of the known substance in the second embodiment.

FIG. 7 is a conceptual graph illustrating a relationship between the concentration of the known substance and Parameter B of the known substance in the second embodiment.

FIG. 8 is a conceptual graph illustrating a relationship between the number of molecules of the known substance and the molecular weight of the known substance in the second embodiment.

FIG. 9 is a graph illustrating the relationship between the concentration of the known substance and the number of molecules and the molecular weight of the known substance in Example 1.

FIG. 10 is a graph illustrating a relationship between the concentration of the known substance and the number of molecules of the known substance in Example 1.

FIG. 11 is a graph illustrating a relationship between the number of molecules of the known substance and the number of molecules and the molecular weight of the known substance.

### BEST MODE FOR CARRYING OUT THE INVENTION

A method of detecting a target substance, comprising:

i) obtaining, with respect to each of plural kinds of known substances each having the same recognition site, a relationship a between a concentration of a known substance and Parameter A of the known substance and a relationship b between the concentration of the known substance and Parameter B of the known substance, and a relationship c between Parameter A of the known substance and Parameter B of the known substance from the relationship a and the relationship b, wherein Parameter A of X is a value derived from a number of molecules of X and Parameter B of X is a value derived from the number and a molecular weight of X;

ii) measuring Parameter A of a target substance contained in a sample and Parameter B of the target substance contained in the sample;

iii) from the relationship c in each of the plural kinds of known substances, Parameter A of the target substance, and Parameter B of the target substance,

determining, when one kind of target substance is contained in the sample, to which one of the plural kinds of the known substances the target substance corresponds; and

determining concentration ratios of plural kinds of the known substances which form the target substance contained in the sample when plural kinds of the target substance are contained in the sample.

### First Embodiment

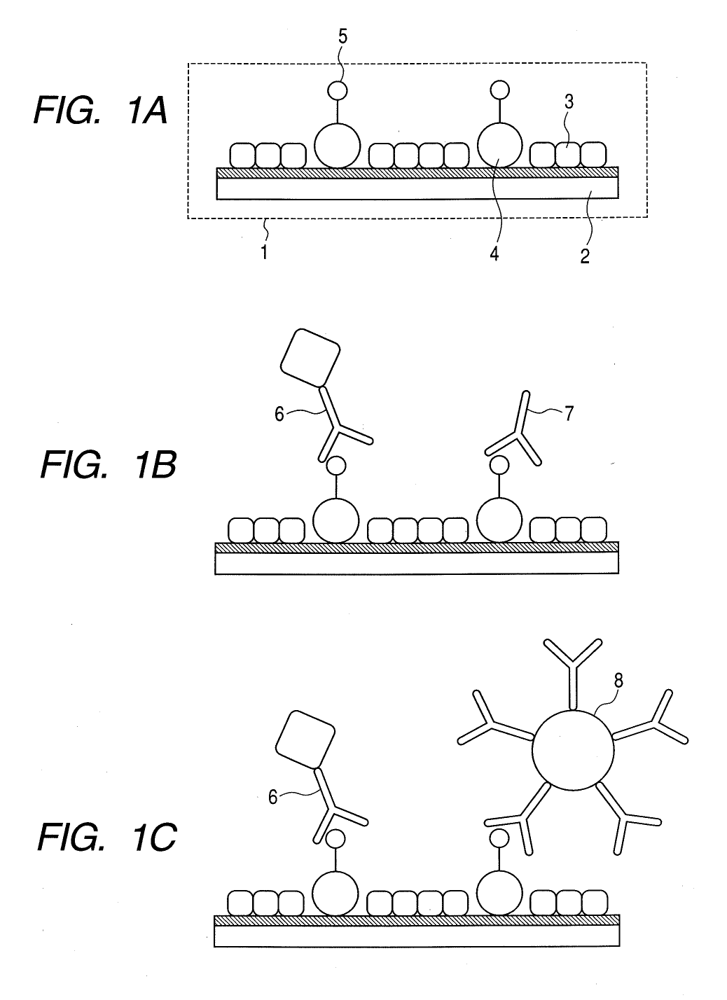

A first embodiment as an example of embodiments of the present invention is described by way of FIGS. 1 to 4.

Note that, in this embodiment, described are an example employing a competition method as a method of obtaining Parameter A, and an example employing a localized surface plasmon resonance method as a method of obtaining Parameter B. In addition, the case where plural kinds of known substances include a known substance-1 and a known substance-2 is described as an example. Note that the molecular weight of the known substance-2 is assumed to be larger than that of the known substance-1 and the binding ability of the substance-1 to a probe is assumed to be higher than that of the substance-2.

Hereinafter, each step is described in detail.

Regarding Step i)

With respect to plural kinds of known substances each having the same recognition site, there are the following stages to be performed: a stage for obtaining a relationship a between a concentration of a known substance and Parameter A of the known substance, a relationship b between the concentration of the known substance and Parameter B of the known substance; and a stage for obtaining, a relationship c between Parameter A of the known substance and Parameter B of the known substance from the relationship a and the relationship b.

FIG. 1A illustrates an example of a detection device 1 that can be used in this example.

The detection device 1 includes a substrate 2, a metal structure 4, and a target substance-capturing body 5.

The substrate 2 functions as a support of the detection device 1. As a material forming the substrate 2, silicon, glass, plastics formed of polystyrene and polymethacrylonitrile, and the like are exemplified. Of those, glass and a plastic formed of polystyrene are preferred. In addition, the substrate may be formed of a plurality of layers. When the substrate is formed of a plurality of layers, an outermost layer is preferably a nonspecific adsorption-preventing film 3. In addition, a layer contacting with the outermost layer (the second layer from the outermost surface) may be formed of ITO, carbon, or the like. Examples of the nonspecific adsorption-preventing film include bovine serum albumin, skimmed milk, and polyethylene glycol.

A plurality of metal structures 4 on the substrate 2 are apart from each other and arranged in line or at random, and induce localized surface plasmon resonance. As a material for the metal structure, gold, silver, copper, platinum, aluminum, or an alloy thereof is preferred. Of those, gold is preferred. In addition, the size of the metal structure preferably falls in a range of 5 nm to 1,450 nm. More preferred size is 50 nm to 450 nm. Any shape of the metal structure is allowable as long as a measurement using the localized surface plasmon resonance can be performed. For example, the metal structure may be a spherical shape, rod-type, acicular, a hollow device, a layered structure formed of different metals from one another, a layered structure formed of a metal and a dielectric substance, a tube-type, and the like. In addition, the metal structure may have, for example, a convexo-concave shape or a projection as long as the measurement using the localized surface plasmon resonance can be performed.

The target substance-capturing body 5 binds to a target substance, a known substance, and a labeled probe specifically and is fixed to a surface of the metal structure. Examples of the combinations of the target substance-capturing body 5 and the target substance include antigen-antibody, enzyme-substrate, hormone-receptor, protein-peptide, sugar chain-sugar chain, sugar chain-antibody, nucleic acid-antibody, and nucleic acid-protein. Note that the case where the combination of the target substance-capturing body 5 and the target substance is denoted by P-Q includes both the case where P represents the target substance-capturing body 5 and Q represents the target substance and the case where Q represents the target substance-capturing body 5 and P represents the target substance.

By using the detection device 1, a known substance-1 7 at each concentration and a labeled probe 6 are reacted competitively.

FIG. 1B illustrates a competitive reaction between the known substance-1 7 and the labeled probe 6. The known substance-1 7 at each concentration and the labeled probe 6 bind to the target substance-capturing body 5 competitively, whereby a complex of the metal structure 4, the target substance-capturing body 5, and the labeled probe 6 and a complex of the metal structure 4, the target substance-capturing body 5, and the known substance-1 7 are formed.

A line 9 in FIG. 2 illustrates the obtained relationship a-(1) between the concentration of the known substance-1 and a signal of the labeled probe. The signal of the labeled probe 6 represented by an ordinate axis in FIG. 2 is a value derived from a number of molecules of the known substance-1 bound to the target substance-capturing body 5 in the competitive reaction. Therefore, the thus obtained calibration curve illustrating the relationship between the concentration of the known substance-1 and the signal obtained from the labeled probe 6 (relationship a-(1)) illustrates a relationship between the concentration of the known substance-1 and Parameter A of the known substance-1.

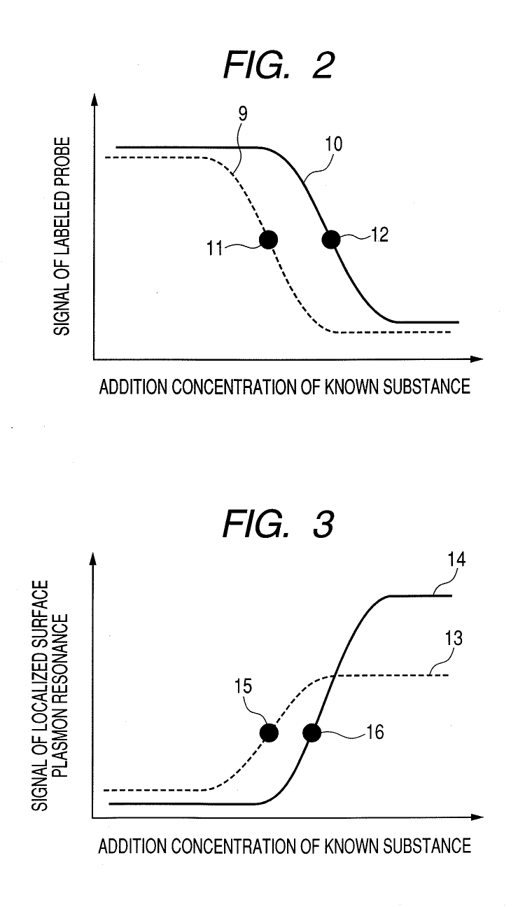

Next, a calibration curve (line 13 in FIG. 3) illustrating a relationship between the concentration of the known substance-1 and a signal derived from localized surface plasmon resonance of the known substance bound to the target-capturing body 5 (relationship b-(1)) is obtained. Measurement of the localized surface plasmon resonance is a measurement method of measuring a refractive index change in the vicinity of the surface of the metal structure 4 incorporated in the detection device 1. The signal of the localized surface plasmon resonance includes information about the number of molecules of the known substance-1 bound to the target substance-capturing body 5 and information about the molecular weight (size) of the known substance-1. Therefore, the calibration curve illustrating a relationship b-(1) between the concentration of the known substance-1 and the signal of the localized surface plasmon resonance of the known substance-1 bound to the target substance-capturing body 5 illustrates a relationship between the concentration of the known substance-1 and a value derived from the number of molecules and the molecular weight of the known substance-1. When the known substance-1 is a multimer, the molecular weight of the substance-1 is defined as a molecular weight of the subunit. For example, when the known substance-1 is a subunit in which b pieces of known substances a are connected, the molecular weight of the known substance-1 is a×b.

Note that when the relationship b-(1) between the concentration of the known substance-1 and the signal of the localized surface plasmon resonance of the known substance-1 bound to the target substance-capturing body 5 is obtained, the signal of the localized surface plasmon resonance and the signal of the labeled probe may be obtained simultaneously or may be obtained separately. In the case of the former, the case where the concentration of the known substance-1 is used as a first concentration, the signal of the labeled probe and the signal of the localized surface plasmon resonance are obtained. After that, the concentration of the known substance-1 is used as a second concentration, and the signal of the labeled probe and the signal of the localized surface plasmon resonance are obtained. In other words, the Parameter A and Parameter B are obtained simultaneously. The latter case includes the following cases: the signal of the labeled probe at each concentration of the known substance-1 (relationship a-(1)) is obtained, and thereafter, the signal of the localized surface plasmon resonance at each concentration of the known substance-1 (relationship b-(1)) is obtained; and the signal of the localized surface plasmon resonance at each concentration of the known substance-1 (relationship b-(1)) is obtained, and the signal of the labeled probe at each concentration of the known substance-1 (relationship a-(1)) is obtained.

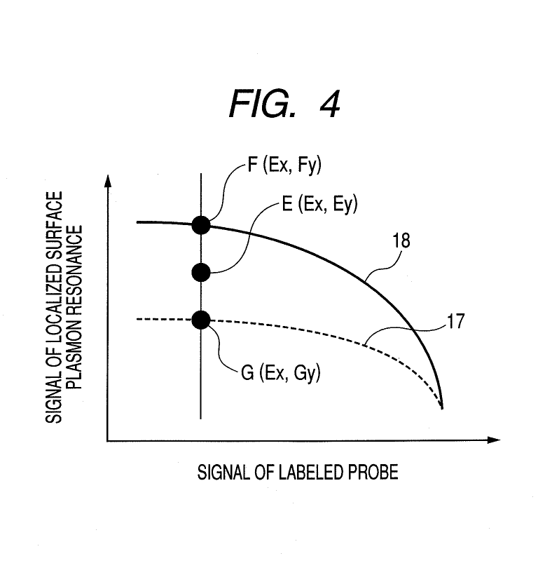

Then, from the thus obtained relationship a-(1) and relationship b-(1), a relationship c-(1) between Parameter A of the known substance-1 and Parameter B of the known substance-1, as shown as the line 17 of FIG. 4, is obtained.

Next, with respect to a known substance-2, a relationship a-(2) between a concentration of the known substance-2 and Parameter A of the known substance-2 is obtained in the same manner as in the known substance-1.

In FIG. 1C, the target probe 6 and the known substance-2 8 bind to the target substance-capturing body 5 competitively, whereby a complex of the metal structure 4, the target substance-capturing body 5, and the labeled probe 6, and a complex of the metal structure 4, the target substance-capturing body 5, and the known substance-2 8 are formed.

The obtained relationship a-(2) between the known substance-2 at each concentration and the signal of the labeled probe is illustrated as the line 10 of FIG. 2. A calibration curve illustrating the relationship a-(2) between the concentration of the known substance-2 and the signal obtained from the labeled probe 6 illustrates a relationship between the concentration of the known substance-2 and Parameter A of the known substance-2 bound to the target substance-capturing body. Note that a point all and a point β12 in FIG. 2 indicate that intensities of the signals of the labeled probes are the same each other and the total number of the known substance-1 bound to the target substance-capturing body 5 and the total number of the known substance-2 bound to the target substance-capturing body 5 are the same each other.

Next, a calibration curve (line 14 in FIG. 3) illustrating a relationship between the concentration of the known substance-2 and the signal derived from the localized surface plasmon resonance of the known substance-2 bound to the target substance-capturing body 5 (relationship b-(2)) is obtained. The calibration curve illustrating the relationship b-(2) between the concentration of the known substance-2 and the signal of the localized surface plasmon resonance of the known substance-2 bound to the target substance-capturing body 5 illustrates a relationship between the concentration of the known substance-2 and Parameter B of the known substance-2. Note that a point γ15 and a point θ16 in FIG. 3 indicate that intensities of the signals of the localized surface plasmon resonance are the same each other and the total molecular weight of the known substance-1 bound to the target substance-capturing body 5 (the molecular weight of the target substance-1×the number of binding) and the total molecular weight of the known substance-2 bound to the target substance-capturing body 5 (the molecular weight of the target substance-2×the number of binding) are the same each other.

From the thus obtained relationships a-(2) and b-(2), a relationship c-(2) between Parameter A of the known substance-2 and Parameter B of the known substance-2, as shown as the line 18 of FIG. 4, is obtained.

Note that the labeled probe 6 specifically binds to the target substance-capturing body 5 incorporated in the detection device 1, and has a labeling site. The labeled probe 6 may be obtained by adding a labeling site to a known substance, or adding a labeling site to a different substance from the known substance. As the labeling site of the labeled probe 6, for example, enzymes such as alkaline phosphatase (ALP) and horseradish peroxidase (HRP), metal fine particles such as a gold colloid and a silver colloid, a magnetic fine particle, a fluorescent dye, an luminescent substrate, a color development substrate, a quantum dot, and the like may be used.

Regarding Step ii)

In a step ii), Parameter A of a target substance contained in the sample and Parameter B of the target substance contained in the sample are measured.

In the step ii), Parameter A of the target substance contained in the sample and Parameter B of the target substance are obtained using the competition method and the localized surface plasmon resonance method in the same manner as in the step i), i.e., the method involving obtaining Parameter A of the known substance and Parameter B of the known substance.

Regarding Step iii)

In a step iii), analyzed are the relationship c-(1) and relationship c-(2) obtained in the step (i), the value derived from the number of molecules of the target substance bound to the target substance-capturing body, and the value derived from the number of molecules and the molecular weight of the target substance bound to the target substance-capturing body, which are obtained in the step ii). According to the analysis, concentration ratios of the known substances (known substances 1 and 2) which form the target substance can be detected quantitatively or qualitatively.

More specifically, in FIG. 4, when a plot of the signals obtained in the step iii) is a point E, the concentration ratios of the known substances which form the target substance can be detected quantitatively or qualitatively from a coordinate of the point E (Ex, Ey), a coordinate of a point F (Ex, Fy), and a coordinate of a point G (Ex, Gy). Here, the point F is a point on the curve illustrating the relationship c-(2) between Parameter A of the known substance-2 and Parameter B of the known substance-2. In addition, the point F has the same X coordinate as that of the point E. The point G is a point on the curve illustrating the relationship c-(1) between Parameter A of the known substance-1 and the Parameter B the known substance-1. In addition, the point G has the same X coordinate as that of the point E. That is, if Ey, which represent a Y coordinate of the point E, satisfies the following formula: Gy<Ey<Fy, it is confirmed qualitatively that the target substance includes both the known substance-1 and the known substance-2.

In addition, if Ey=Gy, it is confirmed that the target substance is the known substance-1, and if Ey=Fy, the target substance is the known substance-2. Further, if Ey satisfies the following formula: Gy<Ey<Fy, from a ratio of |Fy−Ey| to |Gy−Ey|, a ratio of the known substance-1 to the known substance-2, both of which form the target substance, can be quantitatively determined.

### Second Embodiment

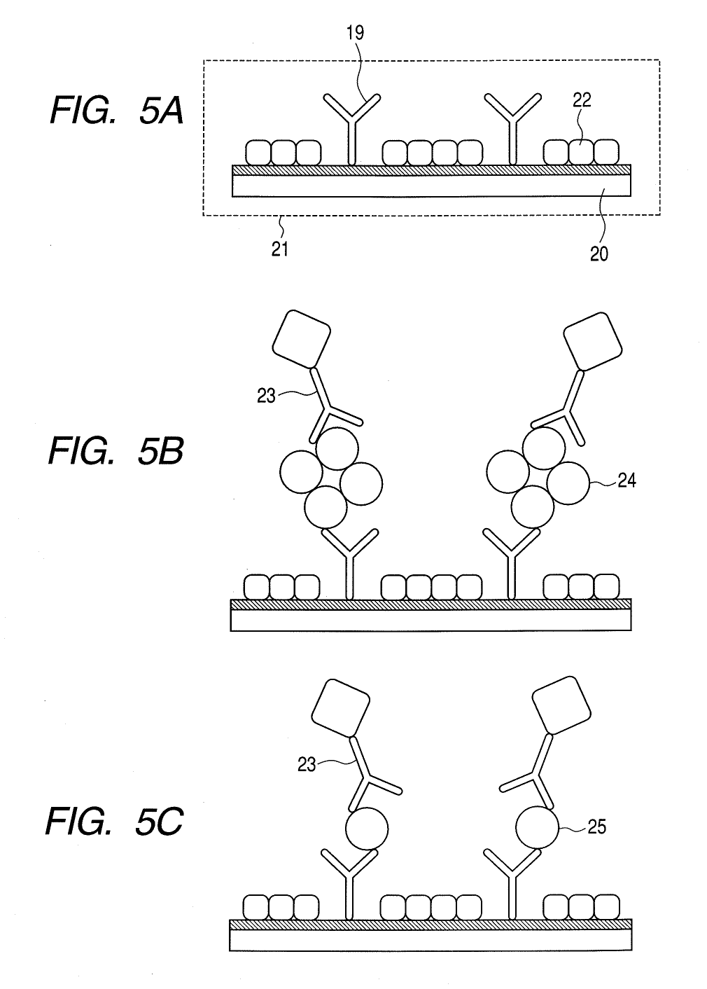

A second embodiment as an example of embodiments of the present invention is hereinafter described by way of FIGS. 5 to 8.

Note that different points between this embodiment and the first embodiment are the following items: in the step i) and ii), a two step sandwich method is used as a measurement method for detecting Parameter A of the known substance and Parameter A of the target substance; reflectometric interference spectroscopy is used as a measurement method for detecting Parameter B of the known substance and Parameter B of the target substance; and the detection device is a device with which reflectometric interference spectroscope can be performed. Except the above-mentioned items, this embodiment is the same as the first embodiment, and only the steps i) and ii) are described. Note that the molecular weight of the known substance-3 is assumed to be larger than that of a known substance-4 and the binding ability of the known substance-3 is assumed to be the same as that of the known substance-4.

Regarding Step i)

With respect to plural kinds of known substances each having the same recognition site, there are the following stages to be performed: a stage for obtaining a relationship a between a concentration of a known substance and Parameter A of the known substance, a relationship b between the concentration of the known substance and Parameter B of the known substance; and a stage for obtaining, from the relationship a and the relationship b, a relationship c between Parameter A of the known substance and Parameter B of the known substance.

FIG. 5A illustrates a detection device 21 in this embodiment.

The detection device 21 includes a substrate 20, and a target substance-capturing body 19.

The substrate 20 has, on a surface, an optical thin film capable of showing an interference color in the reflectometric interference spectroscopy. Note that the substrate may be formed of a plurality of layers. When the substrate is formed of a plurality of layers, an outermost layer is preferably a nonspecific adsorption-preventing film 22.

The target substance-capturing body 19 is fixed on the surface of the substrate 20. The target substance-capturing body 19 binds specifically to the known substance and the target substance. Combinations of the target substance-capturing body 19 and the target substance are the same as in the first embodiment.

A known substance-3 24 at each concentration is bound specifically to the target substance-capturing body 19 fixed on the surface of the substrate 20. Then, obtained is a calibration curve (line 30 of FIG. 7) illustrating a relationship between a concentration of the known substance-3 and a signal derived from an interference color in reflectometric interference spectroscopy of the known substance-3 bound to the target substance-capturing body 19 (relationship b-(3)).

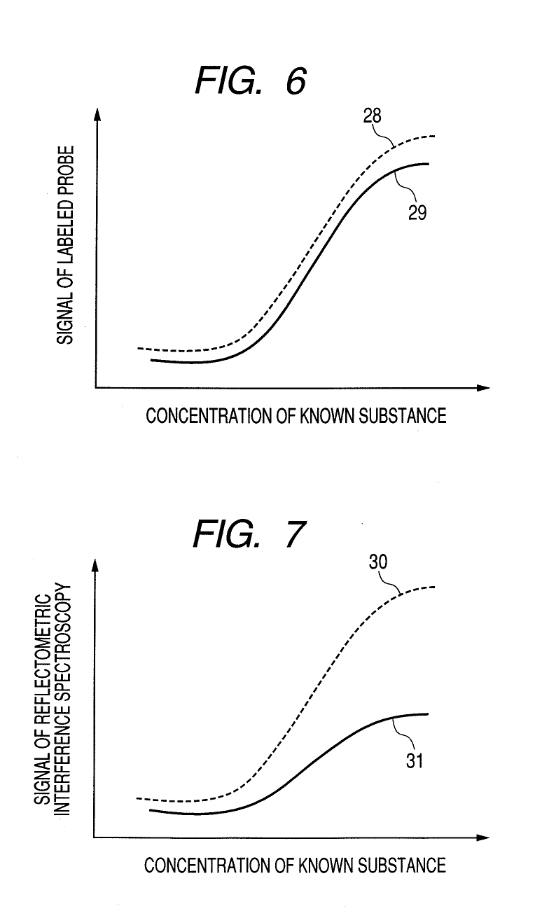

Next, a labeled probe 23 is further bound to a complex of the known substance-3 24 at each concentration and the target substance-capturing body 19. As a result, a complex of the target substance-capturing body 19, the known substance 24, and the labeled probe 23 is formed at each concentration of the known substance-3 24. Then, a signal of the labeled probe 23 is measured, whereby a relationship a-(3), shown as the line 28 of FIG. 6, between the concentration of the known substance-3 and Parameter A of the known substance-3 is obtained.

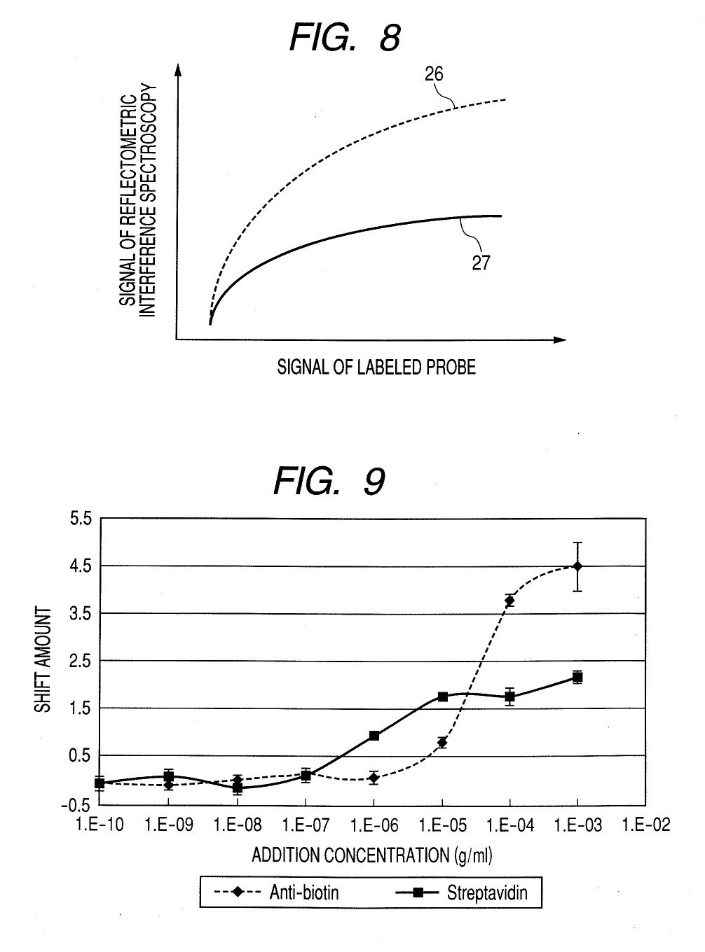

Then, from the thus obtained relationships a-(3) and b-(3), a relationship c-(3) between Parameter A of the known substance-3 and Parameter B of the known substance-3, as shown as the line 26 of FIG. 8, is obtained.

Similarly, with respect to a known substance-4, a known substance-4 25 at each concentration is bound specifically to the target substance-capturing body 19 fixed on the surface of the body 20. Then, obtained is a relationship between a concentration of the known substance-4 and a signal derived from an interference color in reflectometric interference spectroscopy of the known substance-3 bound to the target substance-capturing body 19 (a calibration curve (line 31 in FIG. 7) illustrating relationship b-(4)).

Next, the labeled probe 23 is further bound to a complex of the known substance-4 25 at each concentration and the target substance-capturing body 19. As a result, a complex of the target substance-capturing body 19, the known substance-4 25, and the labeled probe 23 is formed at each concentration of the known substance-4 25. Then, a signal of the labeled probe 23 is measured, whereby a relationship a-(4), shown as the line 29 of FIG. 6, between the concentration of the known substance-4 and Parameter A of the known substance-4 is obtained.

Then, from the thus obtained relationships a-(4) and b-(4), a relationship c-(4) between Parameter A of the known substance-4 and Parameter B of the known substance-4, as shown as the line 27 of FIG. 8, is obtained.

(ii) In the same manner as in the measurement of the step i), Parameter A of the target substance is measured using a two-step sandwich method, and Parameter B of the target substance using the reflectometric interference spectroscopy.

Note that, in the first embodiment, the competition method as a method of measuring Parameter A of the known substance and Parameter A of the target substance, and the localized surface plasmon resonance method as a method of measuring Parameter B of the known substance and Parameter B of the target substance were exemplified for description. In addition, in the second embodiment, the sandwich method as a method of measuring Parameter A of the known substance and Parameter A of the target substance, and the reflectometric interference spectroscopy as a method of measuring Parameter B of the known substance and Parameter B of the target substance were exemplified.

However, in the present invention, any method may be used as a method of measuring Parameter A of the known substance, Parameter A of the target substance, Parameter B of the known substance, or Parameter B of the target substance as long as the method is capable of measuring the number of binding and the concentration of the known substance and the target substance.

As methods of measuring Parameter A of the known substance and Parameter A of the target substance other than the methods used in the first and second embodiments, there are given a radioimmunoassay, an enzyme immunoassay, a fluorescent immunoassay, an enhanced fluorescent immunoassay, a fluorescent quenching immunoassay, a substrate-labeling fluorescent immunoassay, a fluorescent polarization immunoassay, a luminescence immunoassay, a chemiluminescence immunoassay, a chemiluminescent enzyme immunoassay, a bioluminescent enzyme immunoassay, a bioluminescent coenzyme immunoassay, a DNA probe method, an intercalater method, and the like. In addition, an electrochemical measurement disclosed in Japanese Patent Application Laid-Open NO. 2006-133137 as the method of measuring Parameter B of the known substance and Parameter B of the target substance, and an immunoassay using an electrochemical measurement as the method of measuring Parameter A of the known substance and Parameter A of the target substance may be combined.

Further, as methods of measuring the number of molecules and the molecular weights of the known substance and the target substance other than the methods used in the first and second embodiments, there are given a surface plasmon resonance method, a quarts crystal microbalance method, an optical waveguide spectroscopy, an electrochemical measurement method, a Fabry-Perot method, a cantilever method, and the like.

### EXAMPLES

Hereinafter, examples of the present invention are described.

### Example 1

In this example, one kind of a target substance is contained in a sample. This example involves a detection method of determining which one, streptavidin or an anti-biotin antibody, is the target substance. As a method of measuring Parameter B of a known substance and Parameter B of a target substance, a localized surface plasmon resonance was used. In addition, as a method of measuring Parameter A of the known substance and Parameter A of the target substance, a competitive immunoassay was used.

<Production of Detection Device>

A solution containing gold fine particles having an average particle diameter of 100 nm (manufactured by BBI) was diluted to 30% with pure water, and introduced into each well of a 96-well aminated plate (manufactured by SUMITOMO BAKELITE Co., Ltd.). After that, the plate was left to stand at room temperature for 24 hours to fix the gold fine particles to the plate, whereby a substrate having a fine gold structure on a well surface was obtained.

Next, to each well of the substrate, added were 100 μl of 10 μg/ml biotinylated antibody (manufactured by Rockland Immunochemicals, Inc.). The biotinylated antibody was reacted at 4° C. overnight to fix the biotinylated antibody serving as a target substance-capturing body on the substrate surface. After that, 250 μl of 1% casein (manufactured by Techno Chemical Corporation) were added to each well of the substrate, followed by a reaction at 37° C. for 2 hours. As a result, a nonspecific adsorption-preventing film was formed on the substrate surface.

As described above, a detection device for a target substance was produced.

<Competitive Immunoassay>

Streptavidin (manufactured by Funakoshi Corporation) and an anti-biotin (manufactured by ROCKLAND) were used as known substances, and a dilute solution (1×10⁻³ to 10⁻¹¹ g/ml) of each of the known substances was prepared. In addition, a labeled probe as a competitive substance having a concentration of 1×10⁻⁶ g/ml was prepared using an HRP-labeled anti-biotin (manufactured by ROCKLAND).

Next, 50 μl of the dilute solution of the streptavidin were added to each of 48 wells of the detection device and 50 μl of the dilute solution of the anti-biotin were added to each of the other 48 wells of the detection device. After that, 50 μl of the HRP-labeled anti-biotin were added to each of 96 wells of the detection device, followed by being left to stand at 37° for 2 hours.

<Measurement of Localized Surface Plasmon Resonance>

The detection device which underwent the competitive immunoassay was inserted in a microplate reader (manufactured by Thermo Fisher Scientific K.K.) and a localized surface plasmon resonance was then measured.

A spectrum change amount (shift amount) obtained from the measurement of the localized surface plasmon resonance was plotted on an ordinate axis and an addition concentrations of the streptavidin and the anti-biotin were plotted on an abscissa axis, whereby a calibration curve illustrating a relationship a was produced. The calibration curve is illustrated in FIG. 9.

<Enzyme Immunoassay>

A color development kit for peroxidase (manufactured by SUMITOMO BAKELITE Co., Ltd.) was used, and the detection device which underwent the competitive immunoassay was allowed to develop a color. In addition, the color development reaction was measured using the microplate reader.

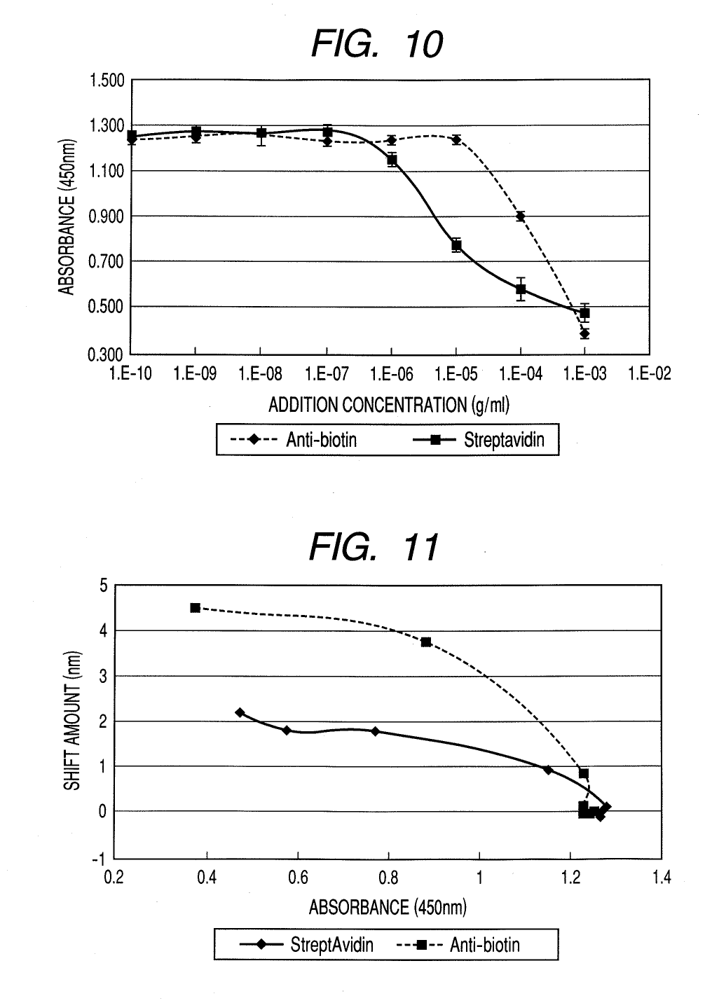

A signal obtained from the color development reaction (absorbance) was plotted on an ordinate axis and an addition concentrations of the streptavidin and the anti-biotin were plotted on an abscissa axis, whereby a calibration curve illustrating a relationship b was produced. The calibration curve is illustrated in FIG. 10.

<Analysis of Signal>

FIG. 11 is an analysis results illustrating a relationship c, which was obtained by plotting the signal obtained from the color development reaction (absorbance) on an abscissa axis and plotting the shift amount obtained from the measurement of the localized surface plasmon resonance on an ordinate axis. In the analysis result of FIG. 11, the abscissa axis shows a signal derived from the number of binding of the known substance bound to biotin which is a target substance-capturing body on the detection device surface. The ordinate axis shows Parameter A and the molecular weight of the known substance bound to biotin. Therefore, it can be confirmed that, from the analysis result, the used anti-biotin has a larger molecule than the streptavidin.

When which one, the streptavidin or the anti-biotin, is the target substance at unknown concentration contained in the sample is identified by using the obtained analysis result illustrating the relationship c as a standard calibration curve, it is possible to determine which one is the target substance by examining which one of the analysis results shown in FIG. 11 is approximate to an analysis of the target substance.

In addition, the target substance can be detected by the following example.

### Example 2

In this example, two kinds of target substances are contained in a sample. This example involves a method of detecting a content ratio between the target substances. In addition, in the detection method, the reflectometric interference spectroscopy is used as a method of measuring Parameter A and the molecular weight of a known substance and the target substances, and a sandwich immunoassay is used as a method of measuring the number of molecules of the known substance and the target substances.

<Production of Detection Device>

A silicon wafer (4 cm×4 cm) on which a silicon nitride is formed into a film and a PMMA (4 cm×4 cm) in which 16 wells each having a diameter of 7 mm are formed are bonded each other. Next, γ-aminopropyl triethoxy silane is applied to wells, whereby amino groups are introduced in the wells. The resultant is used as a substrate.

Next, 100 μl of a mixture solution containing a blood group A antigen (Dextra Laboratories) and glutaraldehyde are added to each well of the substrate to fix the antigen as a target substance-capturing body on the substrate surface by chemical crosslinking. After that, 250 μl of 3% skim milk (manufactured by DIFCO) are added to each well of the substrate, followed by a reaction at 37° C. for 2 hours. As a result, a nonspecific adsorption-preventing film is formed on the substrate surface.

As described above, a detection device for a target substance is produced.

<Sandwich Immunoassay>

As the known substances, an IgG-type anti-A antibody (manufactured by GeneTex, Inc.) and an IgM-type anti-A antibody (manufactured by GeneTex, Inc.) are used, whereby a dilute solution (1×10⁻⁴ to 10⁻¹¹ g/ml) of each known substance is prepared. In addition, a HRP-labeled IgG, IgA, or IgM antibody (manufactured by Acris Antibodies) is used as a sandwich antibody.

First, each target substance is added to the detection device, followed by a reaction at 37° C. for 2 hours. Then, signals are obtained by the reflectometric interference spectroscopy. After that, 100 μl of sandwich antibodies are added to each well, followed by a reaction at 37° C. for 2 hours.

<Reflectometric Interference Spectroscopy>

A reflection spectrum is measured by the immunoassay and using a biosensor array system (manufactured by Fluidware Technologies Inc.).

Then, the spectrum change amount (shift amount) obtained from the reflectometric interference spectroscopy is plotted on an ordinate axis and addition concentrations of the IgG-type anti-A antibody and the IgM-type anti-A antibody are plotted on an abscissa axis, whereby a calibration curve illustrating a relationship a between the concentration of the known substance and Parameter A and the molecular weight of the known substance is obtained.

<Enzyme Immunoassay>

A color development kit for peroxidase (manufactured by SUMITOMO BAKELITE Co., Ltd.) is used, and the detection device which underwent the sandwich immunoassay is allowed to develop a color. In addition, the color development reaction is measured using a microplate reader (manufactured by PerkinElmer).

The signal obtained from the color development reaction (absorbance) is plotted on an ordinate axis and addition concentrations of the IgG-type anti-A antibody and the IgM-type anti-A antibody are plotted on an abscissa axis. Then, produced is a calibration curve illustrating a relationship b between the concentration of each known substance and Parameter A of the known substance.

<Signal Analysis>

The signal obtained from the color development reaction (absorbance) is plotted on an abscissa axis and the shift amount obtained from the reflectometric interference spectroscopy is plotted on an ordinate axis. Then, obtained is a standard calibration curve illustrating a relationship c between Parameter A of each known substance and Parameter A and the molecular weight of each known substance.

Finally, the sample containing the target substances is used and each signal is obtained by the reflectometric interference spectroscopy and the color development reaction. Then, shift amounts at the same absorbance (the shift amounts each obtained by the reflectometric interference spectroscopy) are compared, whereby the content ratio between IgM and IgG, which are target substances in the sample, can be obtained.

This application claims the benefit of Japanese Patent Application No. 2007-328716, filed Dec. 20, 2007, which is hereby incorporated by reference herein in its entirety.

1. A method of detecting a target substance, comprising:
i) obtaining, with respect to each of plural kinds of known substances each having the same recognition site, wherein the target substance corresponds to one of the plural kinds of the known substances, a relationship a between a concentration of a known substance and Parameter A of the known substance and a relationship b between the concentration of the known substance and Parameter B of the known substance, and a relationship c between Parameter A of the known substance and Parameter B of the known substance from the relationship a and the relationship b, wherein Parameter A of X is a value derived from a number of molecules of X and Parameter B of X is a value derived from the number and a molecular weight of X;
ii) measuring Parameter A of a target substance contained in a sample and Parameter B of the target substance contained in the sample;
iii) from the relationship c in each of the plural kinds of known substances, Parameter A of the target substance contained in the sample, and Parameter B of the target substance contained in the sample,
determining, when one kind of target substance is contained in the sample, to which one of the plural kinds of the known substances the target substance corresponds; and
determining concentration ratios of plural kinds of the known substances which form the target substance contained in the sample when plural kinds of the target substance are contained in the sample.

2. A method of detecting a target substance according to claim 1, wherein Parameter B is measured by a localized surface plasmon resonance method.

3. A method of detecting a target substance according to claim 1, wherein Parameter A is measured by a competition method.

4. A method of detecting a target substance, comprising:
i) obtaining, with respect to each of plural kinds of known substances each having the same recognition site, a relationship a between a concentration of a known substance and Parameter A of the known substance and a relationship b between the concentration of the known substance and Parameter B of the known substance, and a relationship c between Parameter A of the known substance and Parameter B of the known substance from the relationship a and the relationship b, wherein Parameter A of X is a value derived from a number of molecules of X and Parameter B of X is a value derived from the number and a molecular weight of X;
ii) measuring Parameter A of a target substance contained in a sample and Parameter B of the target substance contained in the sample;
iii) from the relationship c in each of the plural kinds of known substances, Parameter A of the target substance in the sample, and Parameter B of the target substance in the sample,
determining a condition of the target substance in the sample.

5. A method of detecting a target substance according to claim 4, wherein the target substance is a single kind, and
at determining the condition of the target, to which of the plural kinds of the known substances the target substance corresponds is determined.

6. A method of detecting a target substance according to claim 4, wherein the target substance is plural kinds, and
at determining the condition of the target, a ratio of concentrations of the target substances is determined.
